(19) United States
(12) Patent Application Publication
(10) Pub. No.: US 2006/0068389 A1
(43) Pub. Date: Mar. 30, 2006

(54) Methods and compositions for inhibiting hiv replication

(76) Inventor: David B Weiner, Merion, PA (US)

Correspondence Address: COZEN O'CONNOR, P.C., 1900 MARKET STREET, PHILADELPHIA, PA, 19103-3508, US

(21) Appl. No.: 10/515,327

(22) PCT Filed: May 23, 2003
(86) PCT No.: PCT/US03/16524

§ 371 Date: Nov. 22, 2004

Related U.S. Application Data
(60) Provisional application No. 60/383,409, filed on May 24, 2002.

(51) Int. Cl. C12Q 1/70; C12Q 1/68

(52) U.S. Cl. 435/6; 435/5

(57) ABSTRACT

Pharmaceutial composition comprising compounds and/or compositions useful to inhibit HIV replication are disclosed. Methods of treating individuals infected with HIV are disclosed. Methods of preventing HIV infection in high-risk individuals are disclosed.

### FIELD OF THE INVENTION

The present invention relates to pharmaceutical compositions that inhibit or prevent HIV replication, to methods of treating individuals who are infected with human immunodeficiency virus (HIV) infected, and to methods of preventing HIV infection in individuals who are exposed to HIV.

### BACKGROUND OF THE INVENTION

HIV is a lentivirus whose genome contains only about 9-11 kb of genetic material and less than 10 open reading frames. HIV possesses a collection of small, positive strand open reading frames that encode 1-2 exon genes whose protein products regulate various aspects of the virus' life cycle. Some of these genes are genetic transactivating factors that are necessary for virus replication in all permissive cell types.

The progression from HIV infection to AIDS is in large part determined by the effects of HIV on the cells that it infects, including CD4⁺ T lymphocytes and macrophages. Cell activation, differentiation and proliferation in turn regulate HIV infection and replication in T cells and macrophages. Gallo, R. C. et al. (1984) Science 224:500; Levy, J. A. et al., (1984) Science 225:840; Zack, J. A. et al. (1988) Science 240:1026; Griffin, G. E. et al., (1988) Nature 339:70; Valentin, A. et al. (1991) J. AIDS 4:751; Rich, E. A. et al., (1992) J. Clin. Invest. 89:176; and Schuitemaker, H. et al. (1992) J. Virol. 66:1354. Cell division per se may not be required since HIV and other lentiviruses can proliferate in non-proliferating, terminally differentiated macrophages and growth-arrested T lymphocytes. Rose, R. M. et al. (1986) Am. Rev. Respir. Dis. 143:850; Salahuddin, S. Z. et al. (1986) Blood 68:281; and Li, G. et al. (1993) J. Virol. 67:3969. HIV infection of myeloid cell lines can result in a more differentiated phenotype and increase the expression of factors such as NF-KB that are necessary for HIV replication. Roulston, A. et al. (1992) J. Exp. Med. 175:751; and Chantal Petit, A. J. et al. (1987) J. Clin. Invest. 79:1883.

Since the demonstration in 1987 that the small open reading frame within HIV-1 designated R encodes a 15 KD protein (Wong-Staal, F., et al., (1987) AIDS Res. Hum. Retroviruses 3:33-39), there has been a growing body of literature regarding the function of the viral protein R (Vpr). The ability of lentiviruses, including HIV, to replicate in non-proliferating cells, particularly in macrophages, is believed to be unique among retroviruses. It is significant that several lentiviruses contain a vpr-like gene. Myers, G. et al. (1992) AIDS Res. Hum. Retrovir. 8:373. The vpr open reading frame is conserved within all genomes of HIV-1 and HIV-2 and within all pathogenic isolates of simian immunodeficiency virus (SIV) genomes. The evolutionary requirement for economy in design is deemed to require that the presence of vpr in the HIV genome is related to a specific and non-dispensable function in the viral life cycle.

It has been reported that mutations in the vpr gene result in a decrease in the replication and cytopathogenicity of HIV-1, HIV-2, and SIV in primary CD4⁺T lymphocytes and transformed T cell lines. See, e.g., Ogawa, K., et al., (1989) J. Virol. 63:411041-14; Shibata, R., et al. (1990a) J. Med. Primatol. 19:217-225; Shibata, R., et al. (1990b) J. Virol. 64:742-747 and Westervelt, P. et al. (1992) J. Virol. 66:3925, although others have reported that mutated vpr gene had no effect on replication (Dedera, D., et al. (1989) Virol. 63:3205-3208). Importantly, HIV-2 mutated for vpr has been reported unable to infect primary monocyte/macrophages (Hattori, N., et al. (1990) Proc. Natl. Acad. Sci. USA 87:8080-8084). Further, viral replication in macrophages may be almost completely inhibited by antisense ribonucleotides targeting the vpr open reading frame. This, together with the induction of rhabdomyosarcoma cellular differentiation, are deemed to dictate a crucial function for Vpr in HIV pathogenesis.

The Vpr protein is the only HIV-1 regulatory gene product that has been shown to be incorporated into virions. This would normally suggest a structural role for Vpr, but since vpr deleted viruses are able to produce normal virions, this is deemed to be further evidence of a regulatory role for this molecule. The presence of Vpr in virions has been associated with increased replication kinetics in T lymphocytes, and with the ability of HIV to establish productive infection in monocytes and macrophages. The presence of Vpr protein in viral particles means an early function for Vpr during the infection process, following virus penetration and uncoating. This role is considered to involve Vpr interaction with cellular regulatory mechanisms resulting in an increase in cell permissiveness to sustain viral replication processes. See, e.g., Cohen, E. A., et al. 1990a J. Virol. 64:3097-3099; Yu, X. F., et al. (1990) J. Virol. 64:5688-5693.; and, Yuan, X., et al., (1990) AIDS Res. Hum. Retroviruses 6:1265-1271.

U.S. Pat. No. 5,874,225, which is incorporated herein by reference, discloses several activities and characteristics of Vpr including its ability to inhibit cellular proliferation and its ability to associate with protein product encoded by the gag gene. Vpr action can involve the upregulation of cellular elements that enhance viral gene expression, or the down-modulation of cellular inhibitory pathways affecting such viral processes. Such cellular disregulation is consistent with the observation that Vpr is sufficient for the differentiation and cessation in cellular proliferation of rhabdomyosarcoma and osteosarcoma cell lines (Levy, D. N. et al. (1993) Cell 72:541). The ability of a virally associated protein such as Vpr to reinitiate an arrested developmental program is clearly based upon its interaction with other cellular proteins, and since Vpr protein originates within viral particles, it is considered that Vpr must, accordingly, play a role in establishing productive infection.

U.S. Pat. No. 5,780,238, which is incorporated herein by reference, describes the isolation of an approximately 41 KD Vpr cytosolic binding or interacting protein, which has been designated hereafter as Rip-1. As used herein, the term “Rip-1” is meant to refer to the human protein that has an apparent molecular weight of between 40-43 KD, that occurs in the cytoplasm of human cells, that binds to Vpr and that is transported from the cytoplasm to the nucleus when oound to Vpr, either alone or in association with a steroid receptor.

Rip-1 may be co-localized with the T-cell and B-cell transcription factor Nf0B. Vpr and Rip-1 co-elute in an immunoaffinity system, and can be specifically cross-linked to a 58 KD complex. Using peptide and antibody competition, the site of their interaction has been resolved to amino acids 38 to 60 on the Vpr amino acid sequence. Rip-1 has been detected in various cell lines. Rip-1 selectively translocates from the cytosol to the nucleus upon exposure of the cell to Vpr either in a soluble form, or through infection with wild type virus, but not in response to PMA, suggesting a coupling in their regulatory functions. Consequently, the present invention involves the discovery that Rip-1 may be partially responsible for mediating Vpr activity in the human host cell.

U.S. Pat. No. 5,639,598, which is incorporated herein by reference, refers to the discovery that HIV Vpr protein forms a complex with proteins, including Rip-1, in human cells that are in association with, i.e., as a part of or functionally combined with, one or more steroid receptors, especially the glucocorticoid receptor (GR). Inhibitory or antagonist compounds which bind to, or otherwise wholly or partially preclude the formation of a complex involving Vpr and steroid receptors, especially a GR-type receptor, or potentially other components, or one or more steroid receptors alone, prevent or interfere with HIV replication.

Rip-1 functions in association with one or more members of the steroid hormone receptor superfamily, and particularly, in association with one or more members of the glucocorticoid receptor (GR) family, and more particularly, in association with one or more members of the GR-type II receptor family. By “in association with” is meant that Rip-1 is a part of, forms a discrete complex with, or.is functionally interactive or combined with, one or more of said steroid receptors. Thus, the Vpr, Rip-1, and steroid receptor or other component may be chemically and/or physically bound together to form a multi-part complex.

The cellular trafficking characteristics, which have been observed for Rip-1, are consistent with Rip-1 functioning in association with, or even being a member of the steroid hormone receptor superfamily. The glucocorticoid and mineralocorticoid receptors are examples of members of this protein family that are known to translocate from the cytoplasm to the nucleus upon exposure to their ligand. Two types of glucocorticoid receptors have been described. Type I receptors are concentrated in the nucleus even when there is no ligand present. Type II receptors specifically concentrate in the cytoplasm in the absence of ligand, and only translocate to the nucleus in the presence of their appropriate stimulating hormone. The two types of glucocorticoid receptors have high affinity for their specific ligands, and are considered to function through the same transduction pathways. The main functional difference between these two classes of receptors is that the type II receptors are activated by their ligands in such a way that they only transactivate their target cellular protooncogenes in some, but not in all cells. Such cellular specificity is not observed in type I receptors. These observations are consistent with Rip-1 being functionally closely associated with, or actually being a GR-type II molecule.

Glucocorticoid receptors have a number of roles. Glucocorticoid receptors have been shown to act as powerful transactivators. Glucocorticoid receptors have also been shown to operate through the repression of gene expression for particular open reading frames. Glucocorticoid receptor mediated repression is attained by competition for the sites on the DNA molecule that would otherwise be bound by transactivators. An example of the latter is the specific bilateral relationship that has been described for glucocorticoid receptors and c-Jun. In this case, the glucocorticoid receptor represses c-Jun activity, and the opposite is also observed. The phorbol ester PMA has been reported to activate transcription of the AP-1/c-Jun promoter. In addition, glucocorticoids have been shown to counter lymphokine activity as observed by the inhibition of proliferation of a variety of cell lines. This mechanism is deemed to affect immunoregulatory mechanisms in areas such as T cell activation, which is in part mediated by the Jun/AP-1 activity, and its resulting lymphokines. The observation of a cessation in proliferation in different cell lines transfected with Vpr is considered explained by a glucocorticoid receptor mediated pathway, in which Rip-1, alone or in association with one or more steroid receptors or other components, or one or more steroid receptors, acts to bridge viral and cellular activities.

It is also important to note that the glucocorticoid receptors function as a part of a larger multimeric complex. These 330 KD protein clusters comprise a heat shock protein 90 dimer, a heat shock protein 56 unit, and sometimes by a heat shock protein 70 unit (HSP 70), in addition to the specific glucocorticoid receptor molecule; and Rip-1 has been observed in association with this HSP 70. The glucocorticoid receptor polypeptide itself is usually composed of three functional domains arranged in a linear configuration; a hormone binding domain, a DNA binding domain, and a third domain which has been shown to interact with additional cellular proteins, defining the trafficking characteristics of this gene product. It is contemplated that the complex comprising Rip-1, Vpr, and a steroid receptor or other components, may include as an example of the other components, the heat shock protein units described above.

Since Rip-1 in human cells appears to act in conjunction with a member of the steroid hormone receptor superfamily, especially the glucocorticoid receptor family, this may elucidate the manner in which the binding of Vpr to Rip-1 is involved in HIV replication and thus pathogenesis. Accordingly, interactively blocking Rip-1 or a complex including Rip-1 effectively inactivates Vpr and prevents it from converting cells to better HIV replication hosts. The identification of compounds which can inhibit the effects of Vpr and thereby inhibit HIV replication in HIV infected cells is based on the discovery that many of the actions of Vpr are analogous to those of a glucocorticoid. The mechanism of action of Vpr allows for the targeting of that mechanism for active intervention, and thereby the rational design and selection of anti-HIV compounds.

Rip-1 is the first Vpr associating protein which has been identified in accordance with the present invention, but it is possible that other gene products may either interact with Vpr directly, or indirectly through Rip-1 mediated associations. It has also been discovered in accordance with the present invention, that one or more steroid receptors, especially the glucocorticoid, and GR-type II receptors, may form a multi-part complex with, or are otherwise functionally interactive or combined with, Rip-1 and Vpr, whereby Vpr becomes translocated from the cytoplasm to the nucleus of the human host cell, and there plays an essential role in HIV replication.

U.S. Pat. No. 5,780,220, which is incorporated herein by reference, describes the treatment of individuals exposed to or infected with HIV, by administering to such individuals compounds which are steroid hormone receptor antagonists, particularly glucocorticoid receptor antagonists, and more particularly GR-type II receptor antagonists. Such receptor antagonists inhibit or prevent the replicative and other essential functions of Vpr by interactively blocking the Vpr target in human cells. The use of the glucocorticoid receptor antagonist mifeprestone, in the treatment of HIV infected individuals is set forth therein.

There remains a need to identify methods of treating individuals suffering from HIV infection. There remains a need to identify compounds that prevent or inhibit HIV replication in infected cells and thereby are useful for treating individuals suffering from HIV infection. There remains a need to identify methods of treating individuals who have been exposed to HIV to prevent them from becoming HIV infection. There remains a need to identify pharmaceutical compositions useful in such methods.

### SUMMARY OF THE INVENTION

The present invention further relates to pharmaceutical composition comprising: a pharmaceutically acceptable carrier or diluent; and, a compound that inhibits HIV replication, the compound having a structure selected from the group consisting of Formula D1, Formula D2, Formula D3, Formula D4 and pharmaceutically acceptable salts thereof.

The present invention further relates to pharmaceutical composition comprising: a pharmaceutically acceptable carrier or diluent; and, Composition D5.

The present invention further relates to methods of treating an individual who has been infected with HIV. The method comprise the step of administering to the individual an amount of a pharmaceutical composition comprising a pharmaceutically acceptable carrier or diluent, and, a compound that inhibits HIV replication having a structure selected from the group consisting of Formula D1, Formula D2, Formula D3, Formula D4, and pharmaceutically acceptable salts thereof effective to inhibit HIV replication in the individuals.

The present invention further relates to methods of treating an individual who has been infected with HIV. The method comprise the step of administering to the individual an amount of a pharmaceutical composition comprising a pharmaceutically acceptable carrier or diluent, and, Composition D5, and pharmaceutically acceptable salts thereof effective to inhibit HIV replication in the individuals.

The present invention further relates to methods of preventing HIV infection in an individual at an elevated risk of becoming HIV infected. The method comprise the step of administering to the individual a prophylactically effective amount of a pharmaceutical composition that comprises a pharmaceutically acceptable carrier or diluent, and, a compound that inhibits HIV replication having a structure selected from the group consisting of Formula 1, Formula 2, Formula 3, Formula 4, and pharmaceutically acceptable salts thereof effective to inhibit HIV replication.

The present invention further relates to methods of preventing HIV infection in an individual at an elevated risk of becoming HIV infected. The method comprise the step of administering to the individual a prophylactically effective amount of a pharmaceutical composition that comprises a pharmaceutically acceptable carrier or diluent, and, a Composition D5 effective to inhibit HIV replication.

### BRIEF DESCRIPTION OF THE FIGURES

FIG. 1 contains data from Example 6 that demonstrates rescue of proliferation results from the compositions of the invention.

### DETAILED DESCRIPTION OF PREFERRED EMBODIMENTS

The present invention provides pharmaceutical compositions comprising a compound having a structure selected from the group consisting of Formula D1, Formula D2, Formula D3, Formula, D4, and pharmaceutically acceptable salts thereof. The present invention provides pharmaceutical compositions comprising a Composition D5. The present invention provides methods of treating individuals infected with HIV by administering to them a therapeutically effective amount of such compositions. The present invention further provides methods of preventing HIV infection in individuals exposed to HIV, by administering to them a prophylactically effective amount of such compositions.

The present invention is useful to therapeutically treat an individual identified as infected with HIV in order to eliminate, reduce or stabilize viral titer and/or increase or stabilize CD4 + cell counts. The present invention is useful to prophylactically treat a high-risk individual from becoming infected with HIV.

The compounds of the invention may act as steroid hormone receptor antagonists that interactively blocks Rip-1, alone or in association with one or more steroid receptors, or other components, or one or more steroid receptors alone, preventing or inhibiting formation and translocation of the Vpr/Rip-1 and/or steroid receptor or other component complex.

As used herein, the term “high risk individual” is meant to refer to an individual who is suspected of having been exposed to the HIV virus. Such individuals include health care or other individuals who may have accidentally exchanged blood with an HIV-infected individual, such as through an accidental needle stick, injuries that occur during emergency medical care, rescue or arrest and unprotected sexual contact. High-risk individuals can be treated prophylactically before any detection of HIV infection can be made.

As used herein, the term “therapeutically effective amount” is meant to refer to an amount of a compound which produces a medicinal effect observed as reduction or reverse in viral titer and/or and increase or stabilization of CD4+ cell counts when a therapeutically effective amount of a compound is administered to an individual who is infected with HIV. Therapeutically effective amounts are typically determined by the effect they have compared to the effect observed when a composition, which includes no active ingredient, is administered to a similarly situated individual.

As used herein, the term Aprophylactically effective amount” is meant to refer to an amount of a compound which produces a medicinal effect observed as the prevention of HIV infection in an individual when a prophylactically effective amount of a compound is administered to a high risk individual. Prophylactically effective amounts are typically determined by the effect they have compared to the effect observed when a composition, which includes no active ingredient, is administered to a similarly situated individual.

The invention provides novel pharmaceutical compositions comprising antiviral compounds that are inhibitors of HIV replication. The antiviral compounds included in the pharmaceutical compositions of the present invention have a formula selected from the group consisting of Formula D1, Formula D2 and Formula D3, Formula D4, as set forth below, or a pharmaceutically acceptable salt thereof. The invention provides novel pharmaceutical compositions comprising antiviral compositions that inhibit HIV replication. The antiviral compositions included in the pharmaceutical compositions of the present invention include Composition D5, as set forth below, or a pharmaceutically acceptable salt thereof. In some preferred embodiments, the HIV replication inhibitor in the pharmaceutical compositions of the present invention has a formula of Formula 1 as set forth in the section below entitled Formulae. In some preferred embodiments, the HIV replication inhibitor in the pharmaceutical compositions of the present invention has a formula of Formula 2 as set forth in the section below entitled Formulae. In some preferred embodiments, the HIV replication inhibitor in the pharmaceutical compositions of the present invention has a formula of Formula 3 as set forth in the section below entitled Formulae. In some preferred embodiments, the HIV replication inhibitor in the pharmaceutical compositions of the present invention has a formula of Formula 4 as set forth in the section below entitled Formulae. In some preferred embodiments, the HIV replication inhibitor in the pharmaceutical compositions of the present invention is the composition described below as Composition D5 as set forth in the section below.

In some embodiments the method of the invention additionally includes the use of the HIV replication inhibitor compositions of the invention in combination with other methodologies to treat HIV infection. In some embodiments, the HIV replication inhibitor is administered in conjunction with other antiviral agents such as mifepristone, nucleoside and non-nucleoside RT inhibitors (zidovudine, lamivudine, didanosine, abacavir, stavudine, zalcitabine, efavirenz, nevirapine, and delavirdine), protease inhibitors (amprenavir, indinavir, lopinavir, nelfinavir, ritonavir, and saquinavir), and fusion inhibitors (T-20 and T-1249) as well as acyclovir, ganciclovir, foscarnet, interferon alpha-2a, and interferon alpha-2b.

The pharmaceutical compositions comprising HIV replication inhibitor compositions of the present invention may be administered by any means that enables the active agent to reach the agent's site of action in the body of the individual. Pharmaceutical compositions of the present invention may be administered by conventional routes of pharmaceutical administration. Pharmaceutical compositions may be administered parenterally, i.e. intravenous, subcutaneous, intramuscular. In some embodiments, the pharmaceutical compositions are administered orally. Pharmaceutical compositions are administered to the individual for a length of time effective to eliminate, reduce or stabilize viral titer and/or increase or stabilize CD4+ cell counts. When used prophylactically, Pharmaceutical compositions are administered to the individual for a length of time during which monitoring for evidence of infection continues.

Pharmaceutical compositions of the present invention may be administered either as individual therapeutic agents or in combination with other therapeutic agents. They can be administered alone, but are generally administered with a pharmaceutical carrier selected on the basis of the chosen route of administration and standard pharmaceutical practice.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.001 to 1 grams per kilogram of body weight, in some embodiments about 0.1 to 100 milligrams per kilogram of body weight. Ordinarily dosages are in the range of 0.5 to 50 milligrams per kilogram of body weight, and preferably 1 to 10 milligrams per kilogram per day. In some embodiments, the pharmaceutical compositions are given in divided doses 1 to 6 times a day or in sustained release form is effective to obtain desired results.

Dosage forms (composition) suitable for internal administration generally contain from about 1 milligram to about 500 milligrams of active ingredient per unit. In these pharmaceutical compositions the active ingredient will ordinarily be present in an amount of about 0.5-95 by weight based on the total weight of the composition. Generally, multiple administrations are performed.

Pharmaceutical compositions may be formulated by one having ordinary skill in the art with compositions selected depending upon the chosen mode of administration. Suitable pharmaceutical carriers are described in Remington's Pharmaceutical Sciences, A. Osol, a standard reference text in this field, which is incorporated herein by reference.

For parenteral administration, the compound can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and non-aqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. In some embodiments, a parenteral composition suitable for administration by injection is prepared by dissolving 1.5% by weight of active ingredient in 0.9% sodium chloride solution.

According to some embodiments of the present invention, the composition is administered to tissue of an individual by topically or by lavage. The compounds may be formulated as a cream, ointment, salve, douche, suppository or solution for topical administration or irrigation. Formulations for such routes administration of pharmaceutical compositions are well known. Generally, additives for isotonicity can include sodium chloride, dextrose, mannitol, sorbitol and lactose.

In some cases, isotonic solutions such as phosphate buffered saline are used. Stabilizers include gelatin and albumin. In some embodiments; a vasoconstriction agent is added to the formulation. The pharmaceutical preparations according to the present invention are preferably provided sterile and pyrogen free. The pharmaceutical preparations according to the present invention, which are to be used as injectables, are provided sterile, pyrogen free and particulate free.

A pharmaceutically acceptable formulation will provide the active ingredient(s) in proper physical form together with such excipients, diluents, stabilizers, preservatives and other ingredients as are appropriate to the nature and composition of the dosage form and the properties of the drug ingredient(s) in the formulation environment and drug delivery system.

In some embodiments, the invention relates to methods of treating patients suffering from HIV infection. In some embodiments, the invention relates to methods of preventing HIV infection in high-risk individuals.

According to some embodiments of the invention, the patient is treated with other antiviral therapy in conjunction the administration of pharmaceutical compositions according to the invention. The use of multiple therapeutic approaches provides the patient with a broader based intervention.

According to some aspects of the present invention, in combination with administration of the composition that comprises the HIV replication inhibitor, the individual is also administered another agent. In some embodiments, in combination with administration of the composition, the individual additionally receives compositions that comprise mifepristone, nucleoside and non-nucleoside RT inhibitors (lamivudine, didanosine, abacavir, stavudine, zalcitabine, efavirenz, nevirapine, and delavirdine), protease inhibitors (amprenavir, indinavir, lopinavir, nelfinavir, ritonavir, and saquinavir), and fusion inhibitors (T-20 and T-1249) as well as acyclovir, ganciclovir, foscarnet, interferon alpha-2a, and interferon alpha-2b. Other antivirals may also be used delivered according to standard protocols using standard agents, dosages and regimens. In some embodiments, the pharmaceutical compositions contain one or more of the compounds selected from the group consisting of Formula D1, Formula D2, Formula D3, Formula D4, and pharmaceutically acceptable salts thereof. In some embodiments, the pharmaceutical compositions contain one or more of the compounds selected from the group consisting of Formula D1, Formula D2, Formula D3, Formula D4, and pharmaceutically acceptable salts thereof in combination with Composition D5. In some embodiments, the pharmaceutical compositions contain one or more of the compounds selected from the group consisting of Formula D1, Formula D2, Formula D3 Formula D4, and pharmaceutically acceptable salts thereof and at least one additional antiviral selected from the group consisting of: mifepristone, nucleoside and non-nucleoside RT inhibitors (zidovudine, lamivudine, didanosine, abacavir, stavudine, zalcitabine, efavirenz, nevirapine, and delavirdine), protease inhibitors (amprenavir, indinavir, lopinavir, nelfinavir, ritonavir, and saquinavir), and fusion inhibitors (T-20 and T-1249) as well as acyclovir, ganciclovir, foscarnet, interferon alpha-2a, and interferon alpha-2b, together with a pharmaceutically acceptable carrier. In some embodiments, the pharmaceutical compositions contain Composition D5 and at least one additional antiviral selected from the group consisting of: mifepristone, nucleoside and non-nucleoside RT inhibitors (lamivudine, didanosine, abacavir, stavudine, zalcitabine, efavirenz, nevirapine, and delavirdine), protease inhibitors (amprenavir, indinavir, lopinavir, nelfinavir, ritonavir, and saquinavir), and fusion inhibitors (T-20 and T-1249) as well as acyclovir, ganciclovir, foscarnet, interferon alpha-2a, and interferon alpha-2b together with a pharmaceutically acceptable carrier.

The pharmaceutical compositions according to the present invention may be administered as a single doses or in multiple doses. The pharmaceutical compositions of the present invention may be administered either as individual therapeutic agents or in combination with other therapeutic agents. The treatments of the present invention may be combined with conventional therapies, which may be administered sequentially or simultaneously.

In addition to treating HIV-infected individual, the present invention relates to methods of preventing HIV infection in high-risk individuals who, for example, are suspected of having been exposed to the virus.

Additionally, the present invention is particularly useful to prevent recurrence of infection in patients who have been previously diagnosed as HIV positive but show no indication of infection.

Those having ordinary skill in the art can readily identify high-risk individuals. Healthcare workers come into contact with infected blood and suffer needle sticks from syringes used on HIV infected individuals. Surgeons cut themselves during surgery. Lab workers, dentists and dental technicians come into contact with infected blood as do emergency medical and rescue workers and law enforcement officers. Individuals involved in athletics and sexually active individuals can also become exposed to the virus. Once any person comes into contact with infected blood, that individual is at an elevated risk of infection.

The present invention is not limited to any particular theory or mechanism of action and while it is currently believed that the compounds identified herein operate through blocking the steroid hormone receptor complex that comprises Rip-1, such explanation of the mechanism of action is not intended to limit the invention. The present invention is further illustrated by the following examples, which are not intended to be limiting in any way.

### EXAMPLES

### Example 1

Compound D1, Pregna4,6,-diene-3,20-dione, may be obtained from Sigma (St. Louis, Mo.), Catalog number R19,725-4, MDL number MCFD00199858.

### Example 2

Compound D2, 17-α-ethynyl-17-β-hydroxyestr-5(10)-En-3-one, may be obtained from Sigma (St. Louis, Mo.), Catalog number R18,844-1, MDL number MCFD00199015.

### Example 3

Compound D3 may be synthesized using chemical synthesis schemes designed according to Strategies for Organic Drug Synthesis and Design, A Wiley-Interscience Publication, 1998 Lednicer, D, ed. John Wiley & Sons, Inc., New York.

### Example 4

Compound D4 may be synthesized using chemical synthesis schemes designed according to Strategies for Organic Drug Synthesis and Design, A Wiley-Interscience Publication, 1998 Lednicer, D, ed. John Wiley & Sons, Inc., New York.

### Example 5

Composition D5 is a combination of combination of Hydrocotisone Acetate and Zidovudine. Hydrocotisone Acetate may be obtained from Sigma (St. Louis, Mo.), Catalog number H4126. Zidovudine may be obtained from Sigma (St. Louis, Mo.), Catalog number 11546.

### Example 6

Among the putative “accessory genes” of HIV-1 the 96 amino acid viral borne vpr gene product has been described to have several novel and important biological activities. These include cytoplasm to nuclear translocation thus empowering HIV to infect and replicate in non-dividing cells, as well as to function to increase viral replication particularly in monocytes. Along with these viral effects, HIV-1 Vpr induces dramatic biological changes in the target cells of HIV infection including induction of changes in transcription patterns and complete inhibition of proliferation which are collectively termed differentiation. These changes occur in the absence of the expression of other viral gene products and suggested that Vpr mediates its pro-viral effects partially or perhaps solely through modulation of the state of the target cell rather than directly on the virus. The arrested cell replication has been extended to demonstrate that the inhibition of proliferation is specifically at the G2 border of the cell cycle further supporting that Vpr activity is directed at cellular targets. A function for Vpr in modulating the glucocorticoid pathway has recently been described and anti-glucocorticoids negatively regulate Vpr activity. These results demonstrate that the cell contains specific receptor(s) molecule(s) through which Vpr mediates its activity and these molecules have implications for cell biology in general. Studies to define the molecules involved in Vpr activity and the relationships between these observations and Vpr activity in primary cell lines are important in both drug design for HIV and understanding the host cell biology itself as well as in the treatment of cancer.

The Vpr gene of HIV-1 encodes a 15 KD virion associated protein which functions as a regulator of cellular processes linked to the HIV life cycle. We observed the interaction of a 41-kD cytosolic viral protein R interacting protein-1 (rip-1), associated with Vpr in vitro, and co-localizes in cells ex vivo. Rip-1 showed a wide tissue distribution among relevant targets of HIV infection. This protein co-elutes with Vpr in an immunoaffmity system and can be cross-linked with Vpr to form a 58 Kd complex. Interestingly, Vpr and Rip-1 were co-inmunoprecipitated with the human glucocorticoid receptor as part of the activated receptor complex. The Vpr mediated stimulation of Rip-1 nuclear translocation, and the viral complementation accomplished with exogenous Vpr protein was mimicked by GR II stimulating steroids. These effects were inhibited by mifepristone, a well-characterized GR II pathway inhibitor. In addition, the levels of LTR-CAT induction attained by Vpr protein were similarly obtained by GR activating steroids. Vpr protein induced CAT activity in cells transfected with a plasmid that contains CAT linked to HIV-1 LTR, pLTR-CAT, and co-localized with Dexamethasone as to the site of its induction of CAT activity from a panel of HIV-1 LTR CAT deletion mutants. In addition, Vpr stimulated the formation of the DNA binding complex which associates with GRE sequences during the glucocorticoid mediated transactivation process. Together these data directly link the activity of the vpr gene product to the glucocorticoid steroid biochemical pathway.

Accordingly to test if additional anti-glucocorticoids might mediate similar effects, screening assays were developed based on the above Vpr activities which include inhibition of antiviral activity, rescue of the inhibition of cell proliferation and viral transactivation studies. Mifepristone analogues were chosen by SAR analysis.

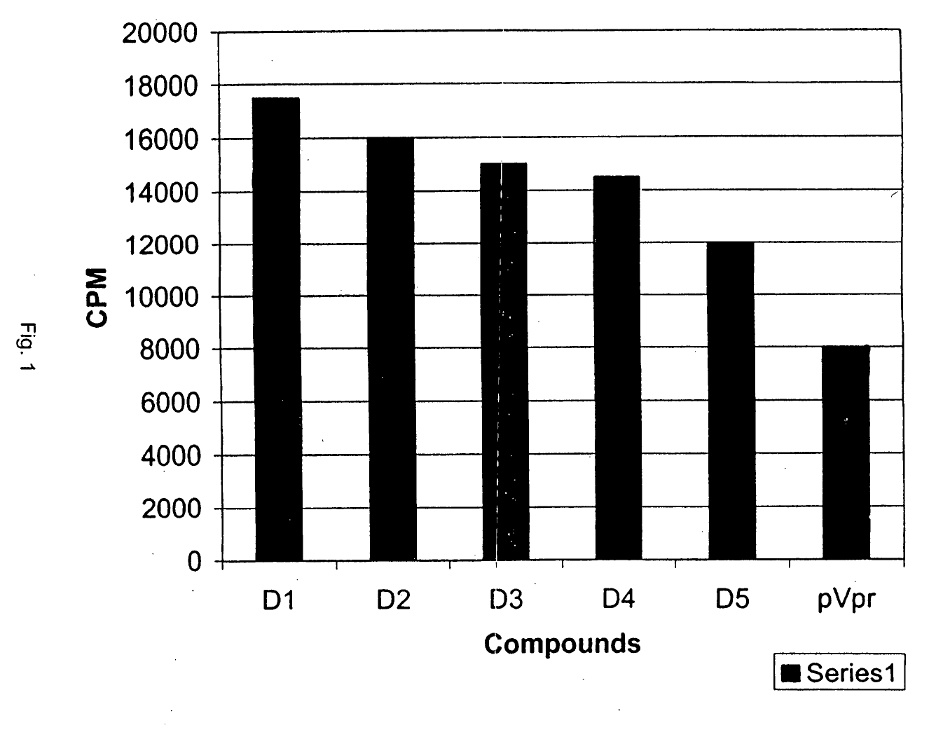

The results depicted in FIG. 1 are typical of the test results seen in multiples assays. Compositions D1, D2, D3, D5 and D6 all exhibited some activity in the above assays. In particular all 5 of the identified compounds rescued Vpr anti-ellular effects (cell cycle arrest). In addition, effects on transactivation were additionally observed. Compound D3 appeared to be the most active in anti-Vpr activity. FORMULAE

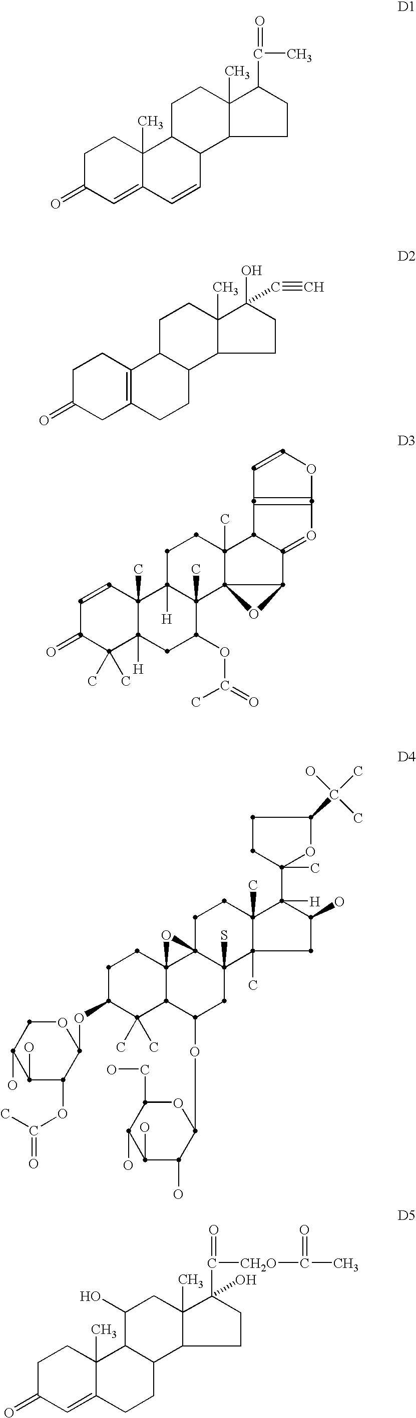

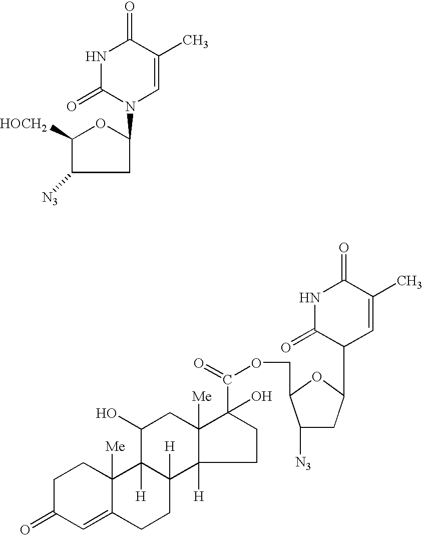

1. A pharmaceutical composition comprising: a pharmaceutically acceptable carrier or diluent; and, a compound having a structure selected from the group consisting of Formula D1, Formula D2, Formula D3, Formula D4 and pharmaceutically acceptable salts thereof and/or Composition D5.

2. The pharmaceutical composition of claim 1 comprising Compound D1.

3. The pharmaceutical composition of claim 1 comprising Compound D2.

4. The pharmaceutical composition of claim 1 comprising Compound D3.

5. The pharmaceutical composition of claim 1 comprising Compound D4.

6. The pharmaceutical composition of claim 1 comprising Composition D5.

7. The pharmaceutical composition of claim 6 further comprising a compound having a structure selected from the group consisting: mifepristone, nucleoside and non-nucleoside RT inhibitors (lamivudine, didanosine, abacavir, stavudine, zalcitabine, efavirenz, nevirapine, and delavirdine), protease inhibitors (amprenavir, indinavir, lopinavir, nelfinavir, ritonavir, and saquinavir), and fusion inhibitors (T-20 and T-1249) as well as acyclovir, ganciclovir, foscarnet, interferon alpha-2a, and interferon alpha-2b.

8. The pharmaceutical composition of claim 1 comprising: a pharmaceutically acceptable carrier or diluent; and, a compound having a structure selected from the group consisting of Formula D1, Formula D2, Formula D3, Formula D4 and pharmaceutically acceptable salts thereof and further comprising a compound having a structure selected from the group consisting: mifepristone, nucleoside and non-nucleoside RT inhibitors (zidovudine, lamivudine, didanosine, abacavir, stavudine, zalcitabine, efavirenz, nevirapine, and delavirdine), protease inhibitors (amprenavir, indinavir, lopinavir, nelfinavir, ritonavir, and saquinavir), and fusion inhibitors (T-20 and T-1249) as well as acyclovir, ganciclovir, foscarnet, interferon alpha-2a, and interferon alpha-2b.

9. A method of treating an individual who is infected with HIV comprising the step of administering to said individual a therapeutically effective amount of a composition according to claim 1.

10. A method of preventing HIV infection in an individual identified as being a high risk individual, the method comprising the step of administering to said individual a prophylactically effective amount of a composition according to claim 1.
